(19) United States
(12) Patent Application Publication
(10) Pub. No.: US 2017/0224942 A1
(43) Pub. Date: Aug. 10, 2017

(54) REBREATHING OXYGEN SUPPLEMENTATION DEVICE

(71) Applicant: Tokitae LLC, Bellevue, WA (US)

(72) Inventors: Michael C. Barbour, Seattle, WA (US); David Richard Bell, Seattle, WA (US); David Gasperino, Lake Forest Park, WA (US); Teo Hurtado, Seattle, WA (US); 3ric Johanson, Seattle, WA (US); Daniel Howard Lieberman, Bellevue, WA (US); Chin Hei Ng, Newcastle, WA (US); Matthew F. Rosen, Seattle, WA (US)

(21) Appl. No.: 15/019,666

(22) Filed: Feb. 9, 2016

(51) Int. Cl. A61M 16/06; A61M 16/00; A61M 16/08

(52) U.S. Cl. CPC A61M 16/0672; A61M 16/0688; A61M 16/0816; A61M 16/0875; A61M 16/0057; A61M 2202/0208

(57) ABSTRACT

In some embodiments, a rebreathing oxygen supplementation device includes an enclosure including an internal surface of a size and shape to entirely cover the nasal region of a patient and including an edge region of a size and shape to reversibly mate with a skin region of the patient surrounding the nasal region. The device can include at least one tubular structure affixed to the enclosure, the tubular structure including a proximal region with a first aperture positioned adjacent to a nostril of the patient and at least one distal region with a second aperture, the tubular structure positioned to permit gas flow between an interior of the enclosure and a region exterior to the tubular structure, and an aperture in the proximal region positioned adjacent to the patient's nostrils.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In some embodiments, a rebreathing oxygen supplementation device includes an enclosure including an internal surface of a size and shape to entirely cover the nasal region of a patient, the internal surface defining an internal volume approximately 25-50% of the tidal volume of the patient, the enclosure including an edge region of a size and shape to reversibly mate with a skin region of the patient surrounding the nasal region.

In some embodiments, a rebreathing oxygen supplementation device includes: an enclosure including an internal surface of a size and shape to entirely cover the nasal region of a patient and including an edge region of a size and shape to reversibly mate with a skin region of the patient surrounding the nasal region; at least one tubular structure affixed to the enclosure, the tubular structure including a proximal region with a first aperture positioned adjacent to a nostril of the patient and at least one distal region with a second aperture, the tubular structure positioned to permit gas flow between an interior of the enclosure and a region exterior to the tubular structure; and an aperture in the proximal region positioned adjacent to the patient's nostrils.

In some embodiments, a rebreathing oxygen supplementation device includes a plurality of structures positioned to form an enclosure including an internal surface of a size and shape to entirely cover the nasal region of a patient and including an edge region of a size and shape to reversibly mate with a skin region of the patient surrounding the nasal region, wherein the plurality of structures include overlapping edges configured to move relative to each other to enlarge or shrink the internal surface of the enclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of an embodiment in use.

FIG. 2 is a schematic of an embodiment in use.

FIG. 3 is a schematic of an embodiment in use.

FIG. 4 is a schematic of an embodiment in use.

FIG. 5 is a schematic of an embodiment.

FIG. 6 is a schematic of an embodiment in use.

FIG. 7 is a schematic of an embodiment in use.

FIG. 8 is a schematic of an embodiment.

FIG. 9 is a schematic of a side view of an embodiment.

FIG. 10 is a schematic of an interior view of an embodiment.

FIG. 11 is a schematic of an embodiment in use.

FIG. 12 is a schematic of an embodiment in use.

FIG. 13 is a schematic of an embodiment in use.

FIG. 14 is a schematic of an embodiment in use.

FIG. 15 is a schematic of an embodiment in use.

FIG. 16 is a schematic of a front view of an embodiment.

FIG. 17 is a schematic of a side view of an embodiment.

FIG. 18 is a schematic of an embodiment in use.

FIG. 19 is a graph of an example test of an embodiment.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

A rebreathing oxygen supplementation device improves the oxygen content in the lungs of a patient by partial recycling of enriched air during exhalation. The device uses an enclosure to partially trap exhaust gases during exhalation, these gasses are held within an enclosure of the device and subsequently can be inhaled on a following inhalation.

In some embodiments, a rebreathing oxygen supplementation device includes an enclosure including an internal surface of a size and shape to entirely cover the nasal region of a patient, the internal surface defining an internal volume approximately 25-50% of the tidal volume of the patient, the enclosure including an edge region of a size and shape to reversibly mate with a skin region of the patient surrounding the nasal region. In some embodiments, a rebreathing oxygen supplementation device includes an enclosure with a shape optimized to capture gas while a patient breathes.

A rebreathing oxygen supplementation device can be designed and fabricated for cleaning and re-use. A rebreathing oxygen supplementation device can be fabricated for sterilization between patients. A rebreathing oxygen supplementation device can be manufactured, for example, from a medical grade plastic material. In some embodiments, a rebreathing oxygen supplementation device includes a design with features and materials that allow for ease of cleaning and re-use. For example, a rebreathing oxygen supplementation device can include surfaces of a size, shape and position to permit easy cleaning for re-use, such as relatively smooth surfaces with minimal crevices or joints that could be difficult to clean. For example, a rebreathing oxygen supplementation device can be fabricated from materials that are stable for medical cleaning, such as durable through autoclaving, bleach treatment, or gas-based sterilization methods.

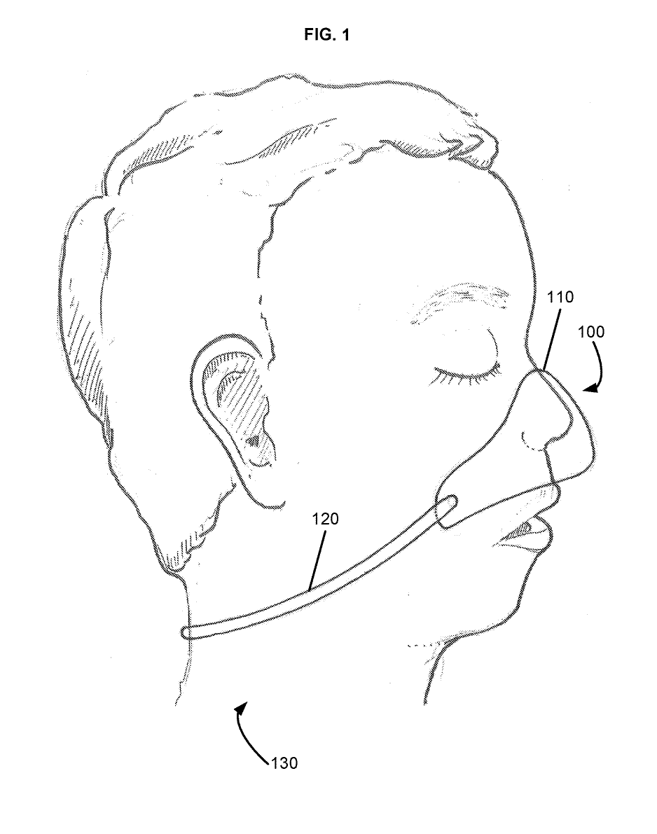

FIG. 1 depicts a side view of a patient with an embodiment of a rebreathing oxygen supplementation device 100. FIG. 1 depicts the rebreathing oxygen supplementation device as transparent for illustration purposes. The rebreathing oxygen supplementation device 100 includes an enclosure 110 which covers the nasal region of the patient 130. As shown in FIG. 1, the enclosure 110 forms a covering over the patient's nose and a section of the face adjacent to the nose, while keeping the mouth region clear. The enclosure 110 includes an edge region of a size and shape to reversibly mate with a skin region of the patient surrounding the nasal region. The rebreathing oxygen supplementation device is of a size, shape and position to not impede vision or obstruct the mouth. Depending on the embodiment, the device may or may not form a tight fit on the nose.

An enclosure of a rebreathing oxygen supplementation device includes an internal surface facing the skin of the patient. The internal surface of the enclosure is of a size and shape to entirely cover the nasal region of the patient. The internal surface of the enclosure defines an internal volume of the enclosure, the internal volume bounded by the internal surface of the enclosure as well as the patient's skin surface. In some embodiments, the internal volume of the enclosure is approximately 25-50% of the patient's tidal volume. The enclosure can be of a size, shape and/or position to optimize the gas capture during breathing. The purpose of the enclosure is to capture the oxygen rich gas that is exhaled from the patient and released from the cannula when the patient is not inspiring, while at the same time maintaining safe carbon dioxide concentrations that avoid hypercapnia conditions.

In the view of FIG. 1, a tube 120 for input of a gas supply, such as air enriched with additional oxygen, is affixed to the lower edge of the enclosure 110 at a position distal to the patient's nose. In some embodiments, a rebreathing oxygen supplementation device includes at least one attachment region for a gas input tube affixed to the enclosure. In some embodiments, the gas input tube includes an end region positioned within the internal volume of the enclosure, the end region of a size, shape and position to direct gas into at least one nostril of the patient. The gas input tube can be affixed to a nasal cannula positioned adjacent to the nostrils of the patient.

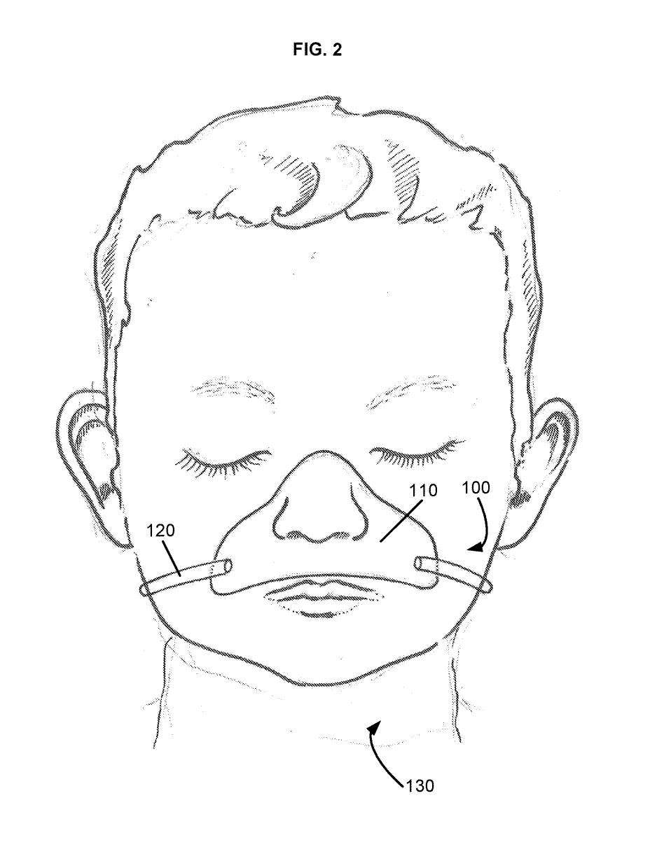

FIG. 2 depicts a frontal view of a rebreathing oxygen supplementation device 100 in use with a patient 130. The rebreathing oxygen supplementation device 100 includes an enclosure 110 covering the nose and adjacent face region of the patient 130. A tube 120 for input of a gas supply is attached to the enclosure 110 and positioned to supply gas to the internal volume of the enclosure 110. In some embodiments, a gas input tube includes an end region positioned within the internal volume of the enclosure, the end region of a size, shape and position to direct gas into at least one nostril of the patient.

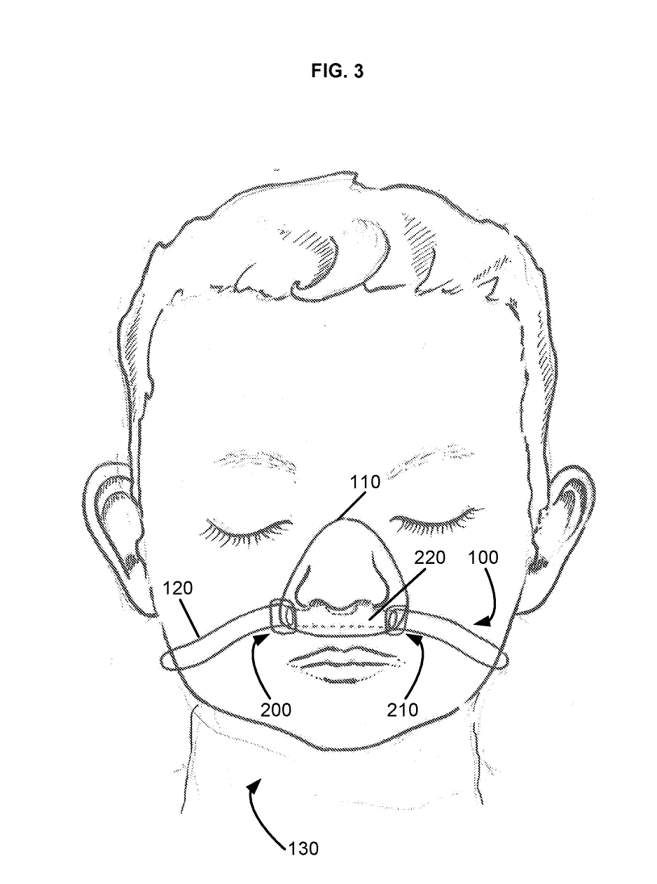

In some embodiments, a rebreathing oxygen supplementation device includes at least one attachment region for a nasal cannula, the at least one attachment region positioned to secure the nasal cannula adjacent to a nostril of the patient. FIG. 3 depicts a frontal view of a rebreathing oxygen supplementation device 100 in use with a patient 130. The rebreathing oxygen supplementation device 100 includes an enclosure 110 with an attachment region 200, 210 for a nasal cannula 220. The attachment region depicted in the embodiment of FIG. 3 includes a first attachment section 200 on one side of the enclosure 110 and a second attachment section 210 on the opposite side of the enclosure 110 relative to the patient's nose.

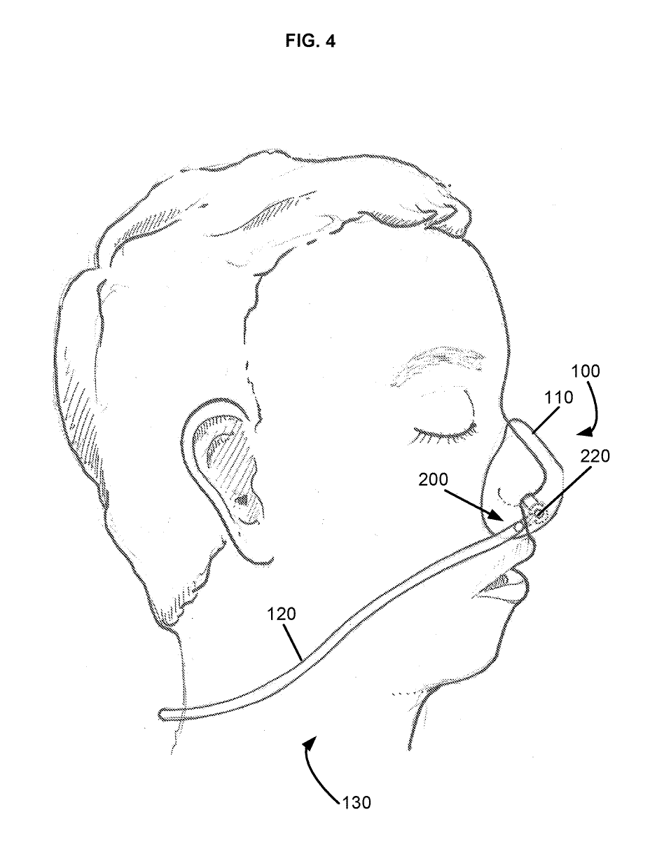

FIG. 4 illustrates, in a side view, an embodiment of a rebreathing oxygen supplementation device 100 in use with a patient 130. The rebreathing oxygen supplementation device 100 includes an enclosure 110 with an attachment region 200 for a nasal cannula 220.

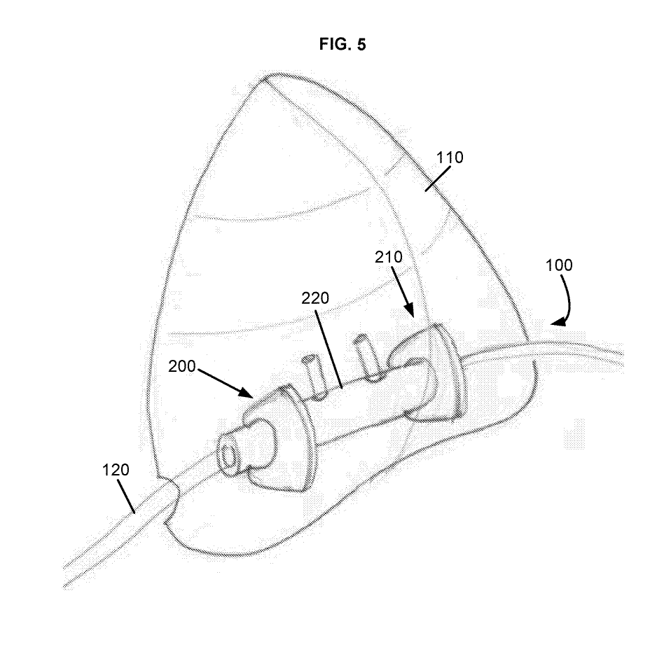

FIG. 5 shows an embodiment of a rebreathing oxygen supplementation device 100 including an enclosure 110 with an attachment region 200, 210 for a nasal cannula 220. The attachment region 200, 210 includes a reversibly attachable bracket of a size, shape and position to hold the nasal cannula 220 in position adjacent to a patient's nose.

For example, the attachment region can include clips or clamps positioned to secure a nasal cannula in place against the internal surface of the enclosure. In some embodiments, an attachment region includes an integrally attached nasal cannula.

In some embodiments, a rebreathing oxygen supplementation device includes: an enclosure including an internal surface of a size and shape to entirely cover the nasal region of a patient and including an edge region of a size and shape to reversibly mate with a skin region of the patient surrounding the nasal region; at least one tubular structure affixed to the enclosure, the tubular structure including a proximal region with a first aperture positioned adjacent to a nostril of the patient and at least one distal region with a second aperture, the tubular structure positioned to permit gas flow between an interior of the enclosure and a region exterior to the tubular structure; and an aperture in the proximal region positioned adjacent to the patient's nostrils.

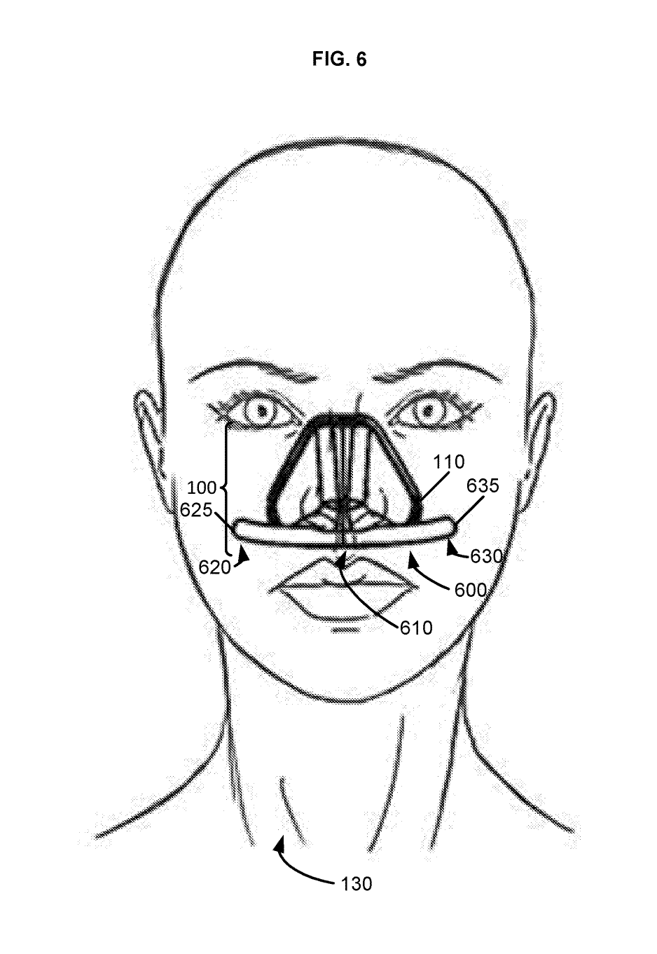

FIG. 6 illustrates an embodiment in a front-facing view in use with a patient. The illustrated rebreathing oxygen supplementation device 100 includes an enclosure 110. The enclosure 110 has an internal surface of a size and shape to entirely cover the nasal region of the patient 130 and including an edge region of a size and shape to reversibly mate with a skin region of the patient 130 surrounding the nasal region. The enclosure 110 also includes a tubular structure 600 affixed to the enclosure 110. The tubular structure 600 includes a proximal region 610 with a first aperture positioned adjacent to a nostril of the patient 130. The tubular structure 600 also includes a first distal region 620 with a first end aperture 625 and a second distal region 630 with a second end aperture 635. The tubular structure 600 is positioned to permit gas flow between an interior of the enclosure 110 and a region exterior to the tubular structure 600.

In some embodiments, the internal volume of the enclosure and the internal volume of the at least one tubular structure in combination form an internal volume of approximately 25-50% of the tidal volume of the patient. In some embodiments a series of internal one way valves or guide vanes could be used to separate oxygen rich gas in the enclosed volume during inhalation and/or exhalation. In some embodiments, this volume captures specific segments of a patient's exhalation, for example, some combination of beginning, middle, or end of exhalation. In some embodiments, a rebreathing oxygen supplementation device includes at least one attachment region for a nasal cannula, the at least one attachment region positioned to secure the nasal cannula adjacent to the patient's nostrils.

Some embodiments include a rebreathing oxygen supplementation device including an enclosure, and an attached tubular structure. In some embodiments a tubular structure includes a first end forming an aperture within the internal surface, and a second end forming an aperture distal to the enclosure. For example, FIG. 6 depicts a rebreathing oxygen supplementation device 100 including a tubular structure 600 with a proximal region 610 and two distal regions 620, 630 with end apertures 625, 635. In some embodiments, a tubular structure includes a single distal region with an end aperture. In some embodiments, a tubular structure includes a single distal region with a plurality of apertures.

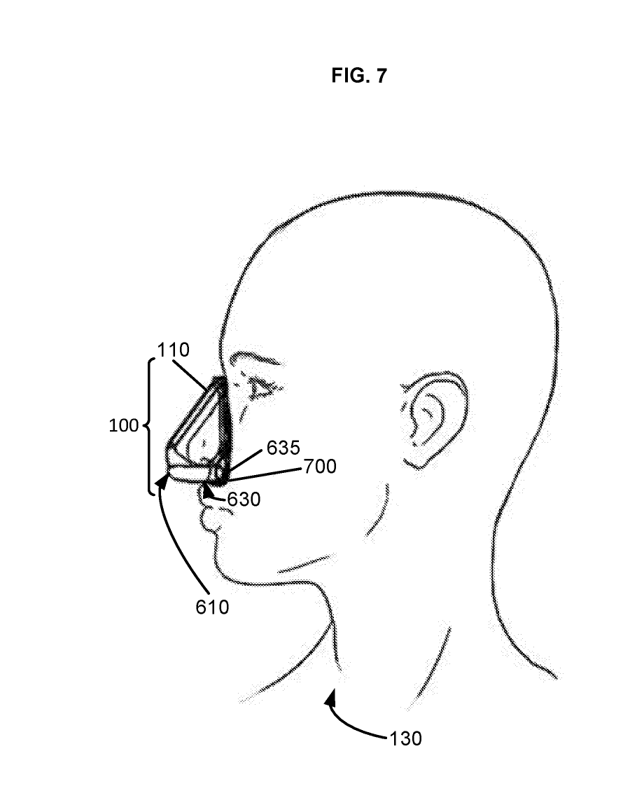

FIG. 7 depicts a rebreathing oxygen supplementation device 100 in use with a patient 130 from a side view. The rebreathing oxygen supplementation device 100 includes an enclosure 110 that fits entirely over the nose and immediate nasal region of the patient. The enclosure 110 includes an edge region 700 of a size and shape to reversibly mate with the skin region of the patient 130 surrounding the nasal region. The edge region 700 includes a lower edge positioned to reversibly mate with the skin region of the patient 130 at a position between the lower nose edge and the top lip of the patient 130. The enclosure also includes a tubular structure 600, which includes a distal region 630 with an end aperture 635.

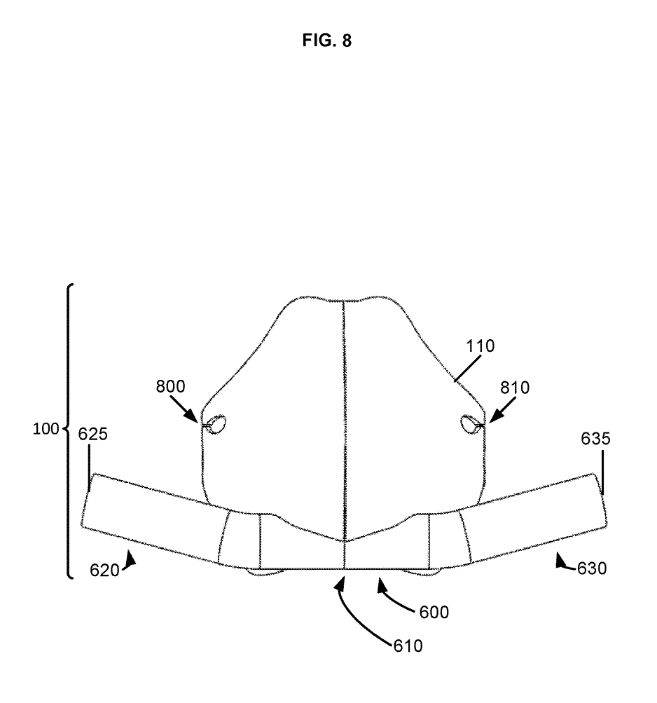

FIG. 8 depicts a schematic of a rebreathing oxygen supplementation device 100 in a frontal view. The rebreathing oxygen supplementation device 100 includes an enclosure 110 affixed to a tubular structure 600. The tubular structure 600 includes a proximal region 610. The tubular structure 600 includes a first distal region 620 with a first end aperture 625 and a second distal region 630 with a second end aperture 635. The enclosure includes a first reversibly attachable bracket 800 and a second reversibly attachable bracket 810, each of a size, shape and position to hold the tube attached to a nasal cannula against the nose of a patient using the rebreathing oxygen supplementation device 100.

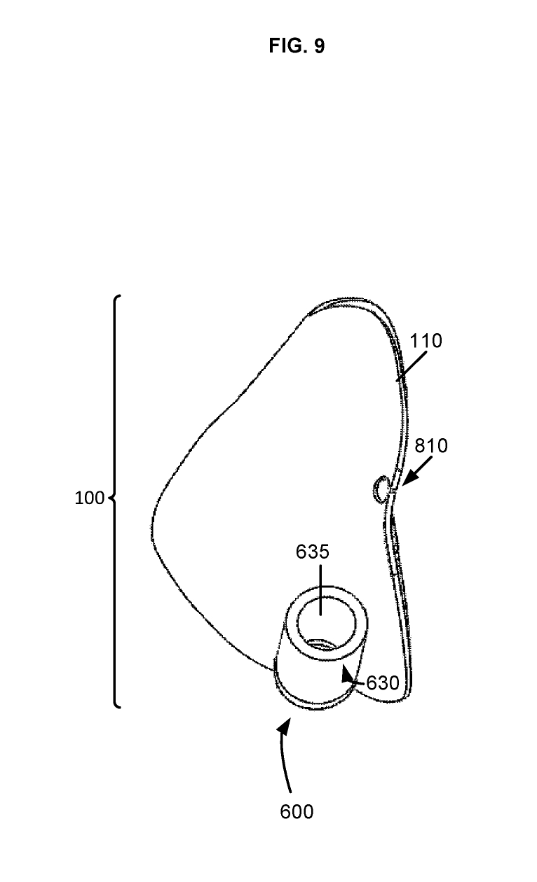

FIG. 9 depicts a schematic of a rebreathing oxygen supplementation device 100 in a side view. The rebreathing oxygen supplementation device 100 includes an enclosure 110 affixed to a tubular structure 600. The tubular structure 600 includes a distal region 630 with an end aperture 635. The enclosure 110 has a reversibly attachable bracket 810 of a size, shape and position to hold the tube attached to a nasal cannula against the nose of a patient using the rebreathing oxygen supplementation device 100.

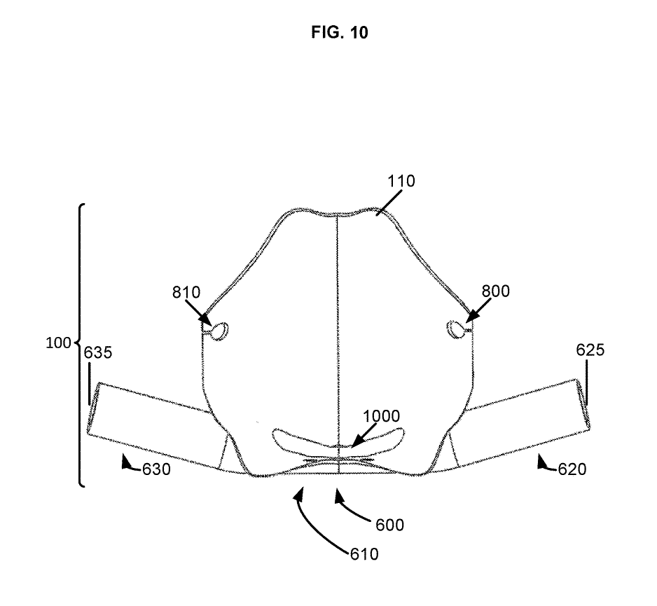

FIG. 10 depicts an embodiment similar to that depicted in FIGS. 8 and 9, from a view of the rebreathing oxygen supplementation device 100 with the surface intended for use adjacent to the patient's face illustrated. The rebreathing oxygen supplementation device 100 includes an enclosure 110 affixed to a tubular structure 600. The tubular structure 600 includes a first distal region 620 with a first end aperture 625 and a second distal region 630 with a second end aperture 635. The enclosure includes a first reversibly attachable bracket 800 and a second reversibly attachable bracket 810, each of a size, shape and position to hold the tube attached to a nasal cannula against the nose of a patient using the rebreathing oxygen supplementation device 100. The tubular structure 600 includes a proximal region 610. The proximal region 610 is positioned to be near the nasal passages of a patient during use of the rebreathing oxygen supplementation device 100, for example adjacent to a nasal cannula in place on the patient's nose. The proximal region 610 includes an aperture 1000. The tubular structure 600 includes a center region and a proximal region 610. The proximal region 610 includes an aperture 1000 within an internal surface of the enclosure 110. The aperture 1000 is positioned in a region adjacent to the patient's nose during use of the rebreathing oxygen supplementation device 100.

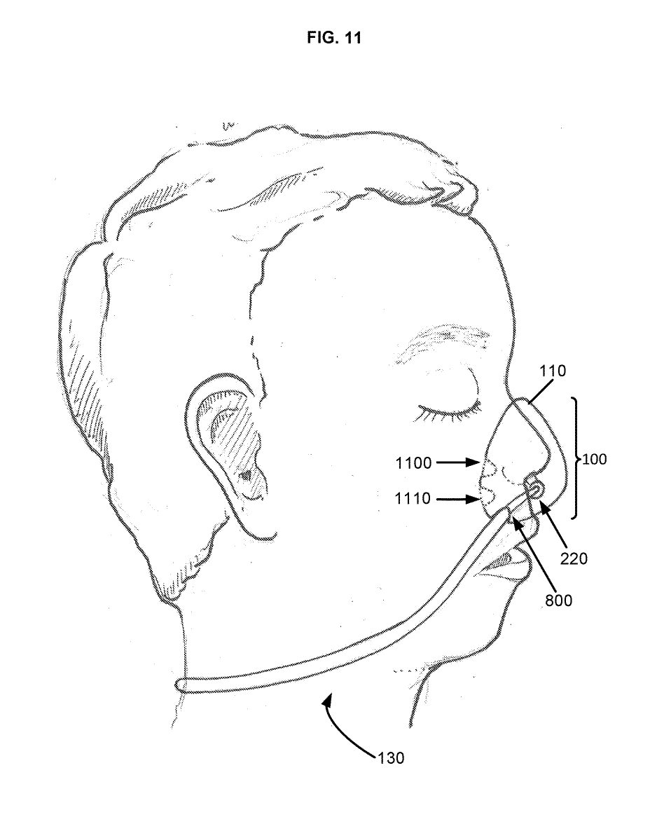

FIG. 11 depicts an embodiment of a rebreathing oxygen supplementation device 100 in use with a patient 130. The rebreathing oxygen supplementation device 100 includes an enclosure 110. A nasal cannula 220 is positioned adjacent to the patient's nostrils under the enclosure 110. A bracket 800 on the enclosure holds the tube of the nasal cannula 220 in position relative to the patient's nose. The enclosure includes a first breakable region 1100 and a second breakable region 1110, wherein each of the breakable regions are of a size and shape to permit removal of a section of an edge of the enclosure 110. Some embodiments include a plurality of breakable regions within the enclosure, each of the breakable regions of a size and shape to position a section of tubing. Each of the breakable regions is of a size and shape to hold a section of tubing in position relative to the patient's face during use of the rebreathing oxygen supplementation device. For example, a region of tubing can be held in place to position the end region of the tubing in position relative to the patient's nose. For example, a region of tubing can be held in place to position an attached nasal cannula in position against the nose of a patient.

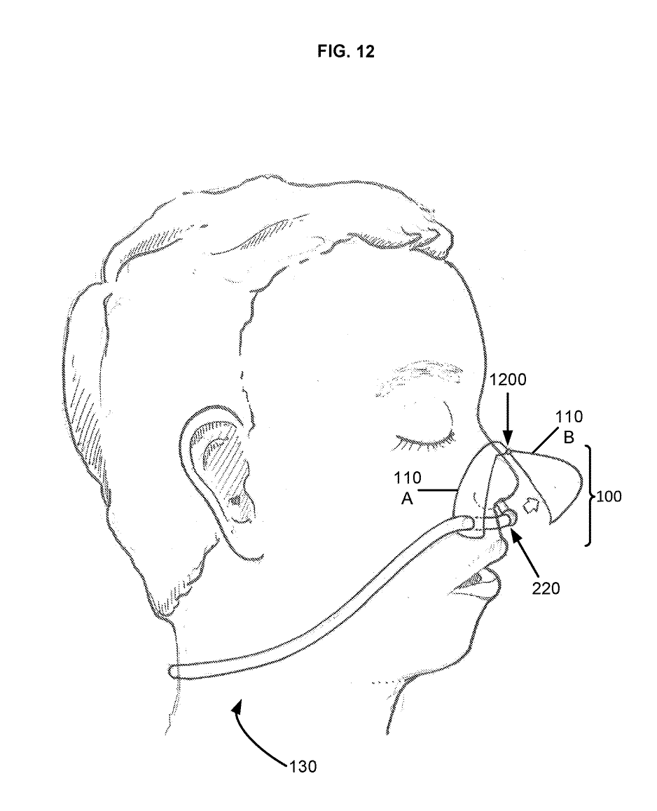

In some embodiments, an enclosure of a rebreathing oxygen supplementation device includes a first section with a first edge, a second section with a second edge of a size and shape to reversibly mate with the first edge, and a hinge mechanism affixed between the first section and the second section, the hinge mechanism positioned to move the first section and the second section relative to each other. FIG. 12 depicts a rebreathing oxygen supplementation device 100 in use with a patient 130. The rebreathing oxygen supplementation device includes an enclosure. The enclosure includes a first section 110A with a first edge, a second section 110 B with a second edge of a size and shape to reversibly mate with the first edge, and a hinge mechanism 1200 affixed between the first section 110 A and the second section 110 B, the hinge mechanism 1200 positioned to move the first section 110 A and the second section 110 B relative to each other. In some embodiments, the enclosure is configured to permit the second section to swing away from the first section while permitting the first section to remain in position relative to the patient's face. The hinge region can, for example, be configured to permit the second section to move sufficiently relative to the first section in order to allow access to the nose of a patient by a healthcare provider. For example, a healthcare provider may wish to adjust the position of the nasal cannula, or clean the nasal region of a patient, without disturbing the entire enclosure.

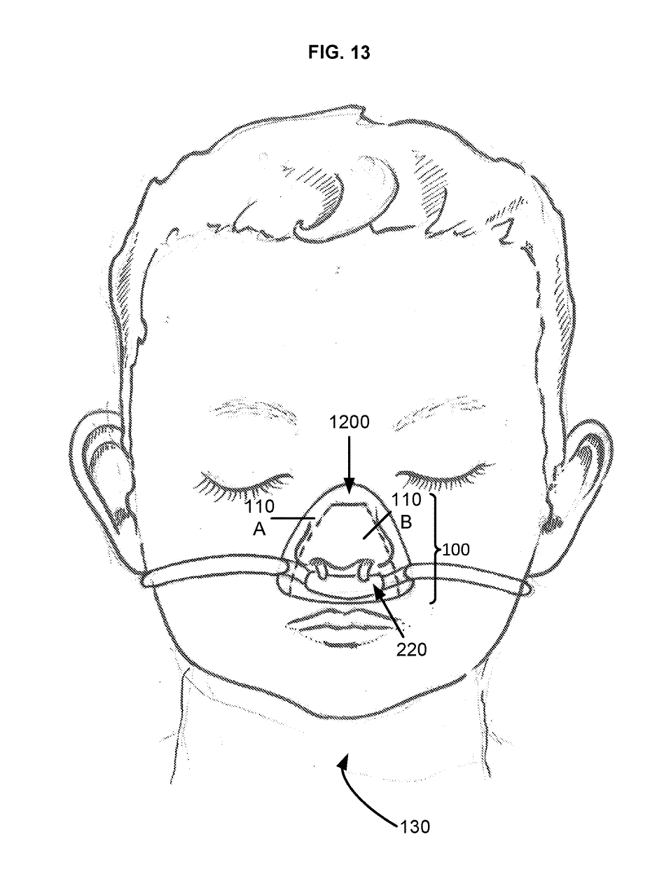

FIG. 13 illustrates an embodiment of a rebreathing oxygen supplementation device 100 including an enclosure with a first section 110 A, a second section 110 B and a hinge region 1200. The hinge region 1200 is affixed with one end to the first section 110 A, and by a second end to the second section 110 B. In the illustration of FIG. 13, the hinge region 1200 is in a closed position. The first section 110 A and the second section 110 B are positioned with their respective edges in proximity to each other, forming an enclosure that retains oxygen from the nasal cannula 220 within the interior volume of the enclosure.

In some embodiments, a rebreathing oxygen supplementation device includes an enclosure with a plurality of substantially flat structures positioned with overlapping edges, the edges configured to move relative to each other to enlarge or shrink the enclosure. In some embodiments, a rebreathing oxygen supplementation device includes a plurality of structures positioned to form an enclosure including an internal surface of a size and shape to entirely cover the nasal region of a patient and including an edge region of a size and shape to reversibly mate with a skin region of the patient surrounding the nasal region, wherein the plurality of structures include overlapping edges configured to move relative to each other to enlarge or shrink the internal surface of the enclosure.

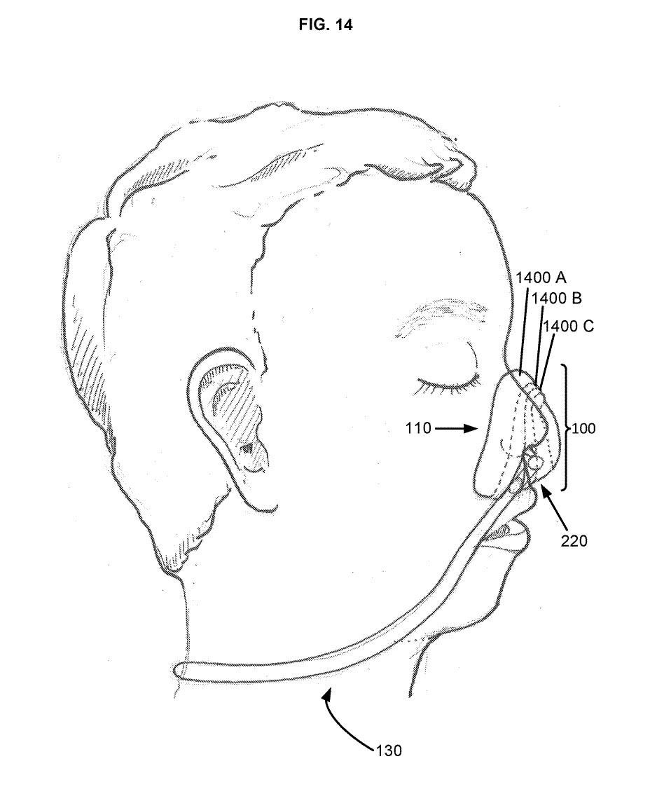

FIG. 14 depicts an embodiment of a rebreathing oxygen supplementation device 100 with an enclosure 110 including a plurality of structures 1400 A, 1400 B, 1400 C, etc. The plurality of structures 1400 A, 1400 B, 1400 C, etc. forming the enclosure are collectively referred to as “structures 1400” in reference to the figures herein. The structure 1400 A of the enclosure 110 closest to the skin region of the patient 130 surrounding the nasal region is of a size and shape to reversibly mate with the skin surface of the patient 130. In some embodiments, the enclosure includes a lower edge positioned to reversibly mate with the skin region of the patient at a position between a lower nose and a top lip of the patient.

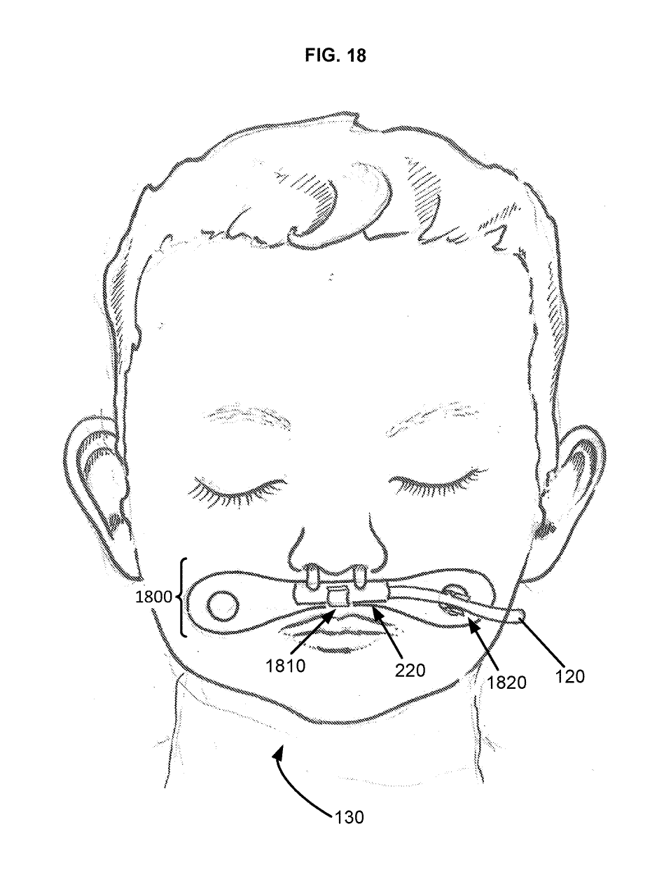

Some embodiments include an attachment region for a nasal cannula, the at least one attachment region positioned to secure the nasal cannula adjacent to the patient's nostrils. The embodiment shown in FIG. 14, for example, includes an integrally attached nasal cannula 220. Some embodiments include an enclosure with a reversibly attachable bracket for a nasal cannula. Some embodiments include a bracket for the nasal cannula, the bracket of a size and shape to reversibly adhere to the patient's face in a region between the bottom of the nose and the top of the mouth (see, e.g. FIG. 18).

The enclosure 110 includes a plurality of structures 1400, with overlapping edges between the structures 1400. For purposes of illustration, the overlapping edges are shown as dotted lines in FIG. 14. The structures 1400 and their respective overlapping edges are configured of a size, shape and position to permit the edges of the structures to move relative to each other. The edges of the structures can, for example, include surfaces that slide relative to each other to enlarge or shrink the internal volume of the enclosure. In some embodiments, the plurality of structures are configured as overlapping sheets.

Some embodiments of an enclosure include a plurality of substantially flat structures positioned with overlapping edges, and further a frame affixed to one or more of the plurality of structures. The frame can be, for example, of a size, shape and position to stabilize the structures relative to each other throughout the motion of the overlapping ends of the structures as the enclosure is enlarged or shrunk. In some embodiments, the frame is flexible. For example, a frame can be manufactured from a bendable plastic material. In some embodiments, a frame is expandable. A frame can, for example, include multiple parts with overlapping edges the slide relative to each other in conjunction with the expansion and contraction of the enclosure.

Enlarging or shrinking the enclosure size can be utilized, for example, by a healthcare professional to customize the fit and size of the internal volume of a rebreathing oxygen supplementation device for a specific patient. A pediatric patient, for example, is likely to need a different internal volume of a rebreathing oxygen supplementation device, and a corresponding different size of enclosure, than an adult patient. In some embodiments, the plurality of structures can be moved relative to each other to define the internal surface of the enclosure with an internal volume of approximately 25-50% of the tidal volume of the patient. In embodiments wherein the enclosure of a rebreathing oxygen supplementation device is being used with patients of different physical sizes (e.g. pediatric as well as adult patients), a medical facility may only need to stock a single, adjustable, model of a rebreathing oxygen supplementation device for use with all patients.

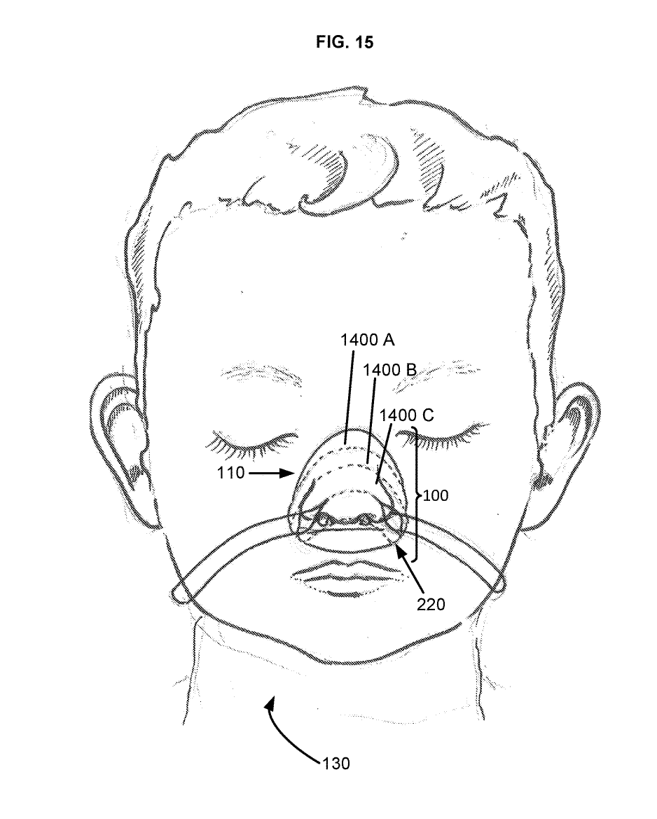

FIG. 15 depicts an embodiment of a rebreathing oxygen supplementation device 100 in use with a patient 130 in a frontal view. The rebreathing oxygen supplementation device 100 includes an enclosure 110 and is used with a nasal cannula 220. The enclosure 110 includes a plurality of structures 1400, with overlapping edges between the structures 1400. The structures 1400 are configured to enlarge the enclosure 110 by reversibly sliding along their overlapping edges when manipulated by a user (e.g. a healthcare worker). Some embodiments include a frame affixed to the structures. Some embodiments include a securing mechanism reversibly affixed to the structures, the securing mechanism positioned and configured to impede relative motion of the structures, for example after the enclosure is correctly sized by a healthcare provider for use with a specific patient.

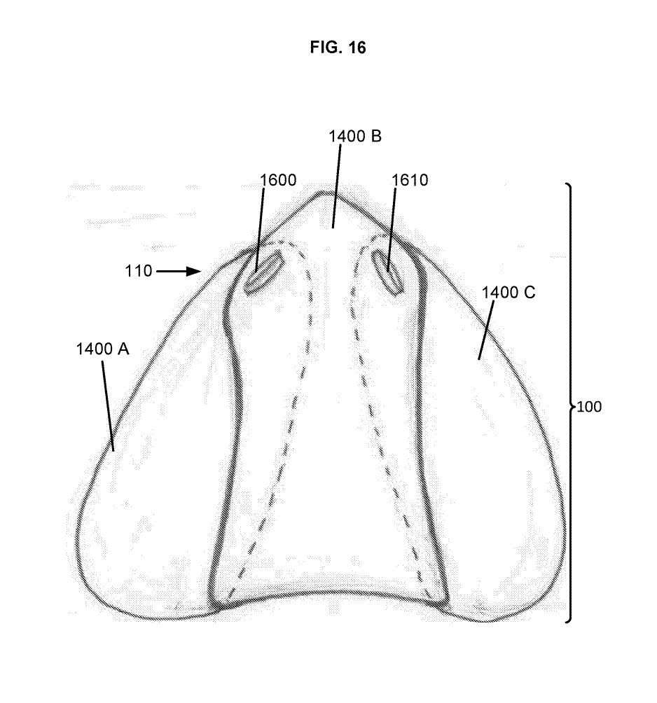

In some embodiments, a rebreathing oxygen supplementation device includes an enclosure with multiple structures that are affixed to each other with a hinge mechanism, the hinge mechanism configured to permit the structures to move relative to each other and therefore to change the internal volume of the enclosure. For example, FIG. 16 depicts a rebreathing oxygen supplementation device 100 that includes an enclosure 110 with three structures 1400 A, 1400 B and 1400 C integrated into the enclosure 110. In the illustrated embodiment, the three structures 1400 A, 1400 B and 1400 C are positioned to form the center 1400 B, right 1400 C and left 1400 A of the enclosure 110 relative to the viewpoint of FIG. 16. The center structure 1400 B has an edge that overlaps with the edge of the left structure 1400 A. A hinge mechanism 1600 is affixed between the center structure 1400 B and the left structure 1400 A. The hinge mechanism 1600 is configured to permit the center structure 1400 B and the left structure 1400 A to move relative to each other to enlarge or shrink the internal volume of the enclosure 110. The center structure 1400 B also has an edge that overlaps with the edge of the right structure 1400 C. The enclosure 110 also includes hinge mechanism 1610, which is affixed between the center structure 1400 B and the right structure 1400 C. The hinge mechanism 1610 is configured to permit the center structure 1400 B and the right structure 1400 C to move relative to each other to enlarge or shrink the internal volume of the enclosure 110.

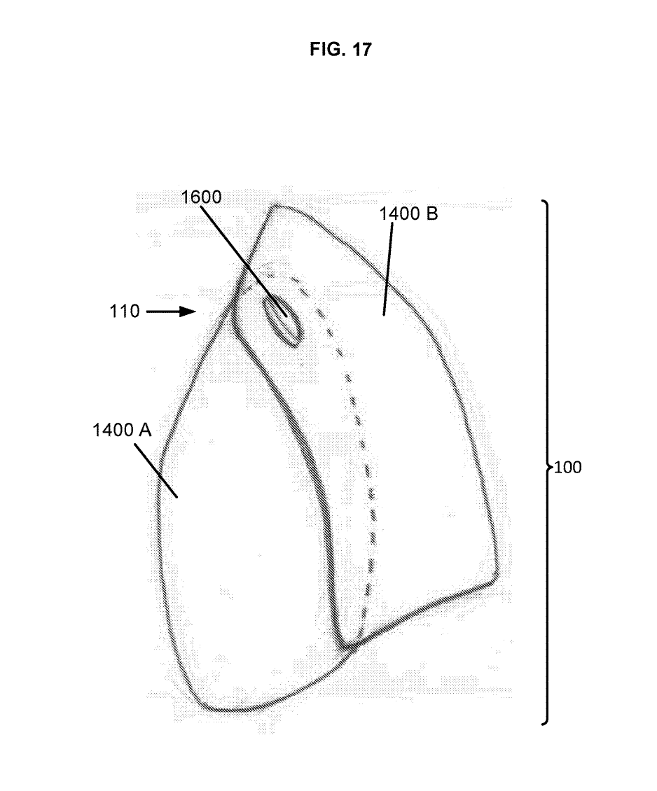

FIG. 17 depicts an embodiment of a rebreathing oxygen supplementation device 100 in a side view. The embodiment illustrated in FIG. 17 includes an enclosure 110 formed with a plurality of structures 1400. The enclosure 110 includes a center structure 1400 B which has an overlapping edge with a side structure 1400 A. For purposes of illustration, the dotted lines represent the overlapping portions of the structures. A hinge mechanism 1600 is attached to both center structure 1400 B and side structure 1400 A. The hinge mechanism 1600 is configured with a size, shape, position and range of motion to permit the center structure 1400 B and side structure 1400 A to move relative to each other. The relative movement of the center structure 1400 B and side structure 1400 A will alter the internal volume of the enclosure 110. In some embodiments, a hinge mechanism includes a ratcheting component configured to minimize slippage of the relative overlap between structures.

Some embodiments include at least one attachment region for a nasal cannula, the at least one attachment region positioned to secure the nasal cannula adjacent to the patient's nostrils. In some embodiments, an attachment region includes a bracket for the nasal cannula, the bracket of a size and shape to reversibly adhere to the patient's face in a region between the bottom of the nose and the top of the mouth.

FIG. 18 depicts a bracket 1800 for a nasal cannula 220 in use with a patient 130. The bracket 1800 is attached to the face of the patient 130 in the region under the nose and above the lips. For example, a bracket could be attached to the face with a reversibly medical adhesive. The bracket is intended for use with a rebreathing oxygen supplementation device covering the nose region of the patient, but the bracket alone is illustrated in FIG. 18 to depict features of the bracket. The nasal cannula 220 is affixed to the bracket 1800 with a first clip 1810. The tube 120 for input of a gas supply attached to the nasal cannula 220 is affixed to the bracket 1800 with a second clip 1820.

### EXAMPLES

### Example 1

An anatomically correct breathing simulator was used to test the fraction of inspired oxygen (FiO2) detected by the simulator relative to the oxygen delivered. The simulator was tested using both a conventional nasal cannula alone as well as with a conventional nasal cannula used in combination with a rebreathing oxygen supplementation device as described herein (see FIGS. 6-10 and associated text).

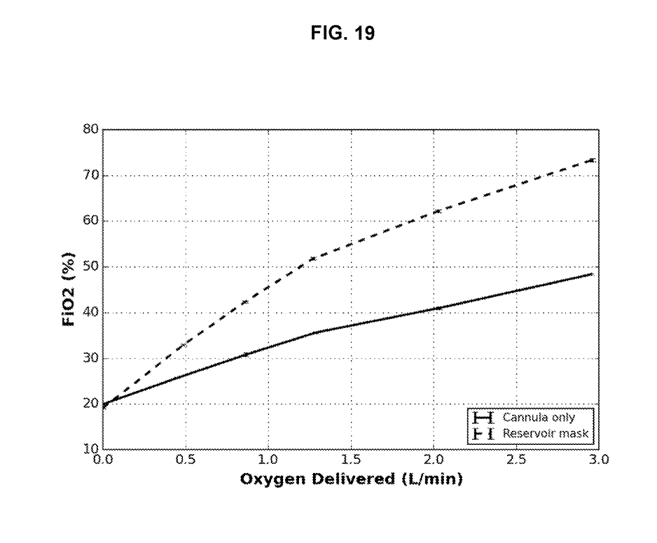

FIG. 19 depicts results from testing with the anatomically correct breathing simulator. The X-axis shows the amount of oxygen delivered via the cannula (in liters per minute) relative to the Y axis depicting the percent fraction of inspired oxygen (FiO2) detected by the simulator. The solid line depicts the results for the cannula alone, while the dotted line depicts the results for the cannula used in combination with a rebreathing oxygen supplementation device (the “reservoir mask”). FIG. 19 indicates that use of a rebreathing oxygen supplementation device in combination with a nasal cannula increases the fraction of inspired oxygen (FiO2) available to the patient relative to a cannula alone. The figure also indicates that this increase in fraction of inspired oxygen (FiO2) is greater with higher levels of oxygen delivery.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A rebreathing oxygen supplementation device comprising:
an enclosure including an internal surface of a size and shape to entirely cover the nasal region of a patient, the internal surface defining an internal volume approximately 25-50% of the tidal volume of the patient, the enclosure including an edge region of a size and shape to reversibly mate with a skin region of the patient surrounding the nasal region.

2. The rebreathing oxygen supplementation device of claim 1, wherein the enclosure comprises:
at least one attachment region for a nasal cannula, the at least one attachment region positioned to secure the nasal cannula adjacent to a nostril of the patient.

3. The rebreathing oxygen supplementation device of claim 2, wherein the at least one attachment region comprises:
a reversibly attachable bracket for the nasal cannula.

4. The rebreathing oxygen supplementation device of claim 2, wherein the at least one attachment region comprises:
an integrally attached nasal cannula.

5. The rebreathing oxygen supplementation device of claim 2, wherein the at least one attachment region comprises:
a bracket for the nasal cannula, the bracket of a size and shape to reversibly adhere to the patient's face in a region between the bottom of the nose and the top of the mouth.

6. The rebreathing oxygen supplementation device of claim 1, wherein the enclosure comprises:
a plurality of breakable regions within the enclosure, each of the breakable regions of a size and shape to position a section of tubing.

7. The rebreathing oxygen supplementation device of claim 1, wherein the enclosure comprises:
at least one attachment region for a gas input tube affixed to the enclosure.

8. The rebreathing oxygen supplementation device of claim 7, wherein the gas input tube comprises:
an end region positioned within the internal volume of the enclosure, the end region of a size, shape and position to direct gas into at least one nostril of the patient.

9. The rebreathing oxygen supplementation device of claim 1, wherein the enclosure comprises:
at least one tubular structure with a first end forming an aperture within the internal surface, and a second end forming an aperture distal to the enclosure.

10. The rebreathing oxygen supplementation device of claim 1, wherein the enclosure comprises:
a tubular structure with a center region forming an aperture within the internal surface, and at least one second end forming an aperture distal to the enclosure.

11. The rebreathing oxygen supplementation device of claim 1, wherein the enclosure comprises:
a first section with a first edge;
a second section with a second edge of a size and shape to reversibly mate with the first edge; and
a hinge mechanism affixed between the first section and the second section, the hinge mechanism positioned to move the first section and the second section relative to each other.

12. The rebreathing oxygen supplementation device of claim 1, wherein the enclosure comprises:
a plurality of substantially flat structures positioned with overlapping edges, the edges configured to move relative to each other to enlarge or shrink the enclosure.

13. The rebreathing oxygen supplementation device of claim 1, wherein the edge region of the enclosure includes a lower edge positioned to reversibly mate with the skin region of the patient at a position between a lower nose and a top lip of the patient.

14. The rebreathing oxygen supplementation device of claim 1, further comprising:
at least one attachment region for a gas input tube affixed to the enclosure; and
a gas emitter affixed to the at least one attachment region, the gas emitter positioned to direct gas into the nasal region of the patient.

15. A rebreathing oxygen supplementation device comprising:
an enclosure including an internal surface of a size and shape to entirely cover the nasal region of a patient and including an edge region of a size and shape to reversibly mate with a skin region of the patient surrounding the nasal region;
at least one tubular structure affixed to the enclosure, the tubular structure including a proximal region with a first aperture positioned adjacent to a nostril of the patient and at least one distal region with a second aperture, the tubular structure positioned to permit gas flow between an interior of the enclosure and a region exterior to the tubular structure; and
an aperture in the proximal region positioned adjacent to the patient's nostrils.

16. The enclosure of claim 15, wherein the internal volume of the enclosure and the internal volume of the at least one tubular structure in combination form an internal volume of approximately 25-50% of the tidal volume of the patient.

17. The enclosure of claim 15, comprising:
at least one attachment region for a nasal cannula, the at least one attachment region positioned to secure the nasal cannula adjacent to the patient's nostrils.

18. The rebreathing oxygen supplementation device of claim 17, wherein the at least one attachment region comprises:
a reversibly attachable bracket for the nasal cannula.

19. The rebreathing oxygen supplementation device of claim 17, wherein the at least one attachment region comprises:
an integrally attached nasal cannula.

20. The rebreathing oxygen supplementation device of claim 17, wherein the at least one attachment region comprises:
a bracket for the nasal cannula, the bracket of a size and shape to reversibly adhere to the patient's face in a region between the bottom of the nose and the top of the mouth.

21. The enclosure of claim 15, comprising:
a plurality of breakable regions within the enclosure, each of the breakable regions of a size and shape to position a section of tubing.

22. The enclosure of claim 15, comprising:
at least one attachment region for a gas input tube affixed to the enclosure.

23. The enclosure of claim 15, comprising:
a plurality of substantially flat structures positioned with overlapping edges, the edges configured to move relative to each other to enlarge or shrink the enclosure.

24. The enclosure of claim 15, wherein the edge region of the enclosure includes a lower edge positioned to reversibly mate with the skin region of the patient at a position between a lower nose and a top lip of the patient.

25. The at least one tubular structure of claim 15, comprising:
at least one tubular structure with a first end forming an aperture within the internal surface, and a second end forming an aperture distal to the enclosure.

26. The at least one tubular structure of claim 15, comprising:
a tubular structure with a center region forming an aperture within the internal surface, and at least one second end forming an aperture distal to the enclosure.

27. The rebreathing oxygen supplementation device of claim 15, further comprising:
at least one attachment region for a gas input tube affixed to the enclosure; and
a gas emitter affixed to the at least one attachment region, the gas emitter positioned to direct gas into the nasal region of the patient.

28. A rebreathing oxygen supplementation device comprising:
a plurality of structures positioned to form an enclosure including an internal surface of a size and shape to entirely cover the nasal region of a patient and including an edge region of a size and shape to reversibly mate with a skin region of the patient surrounding the nasal region, wherein the plurality of structures include overlapping edges configured to move relative to each other to enlarge or shrink the internal surface of the enclosure.

29. The rebreathing oxygen supplementation device of claim 28, wherein the plurality of structures are configured as overlapping sheets.

30. The rebreathing oxygen supplementation device of claim 28, wherein the plurality of structures can be moved relative to each other to define the internal surface of the enclosure with an internal volume of approximately 25-50% of the tidal volume of the patient.

31. The rebreathing oxygen supplementation device of claim 28, comprising:
at least one attachment region for a nasal cannula, the at least one attachment region positioned to secure the nasal cannula adjacent to the patient's nostrils.

32. The rebreathing oxygen supplementation device of claim 31, wherein the at least one attachment region comprises:
a reversibly attachable bracket for the nasal cannula.

33. The rebreathing oxygen supplementation device of claim 31, wherein the at least one attachment region comprises:
an integrally attached nasal cannula.

34. The rebreathing oxygen supplementation device of claim 31, wherein the at least one attachment region comprises:
a bracket for the nasal cannula, the bracket of a size and shape to reversibly adhere to the patient's face in a region between the bottom of the nose and the top of the mouth.

35. The rebreathing oxygen supplementation device of claim 28, comprising:
a frame affixed to one or more of the plurality of structures.

36. The rebreathing oxygen supplementation device of claim 35, wherein the frame is flexible.

37. The rebreathing oxygen supplementation device of claim 35, wherein the frame is expandable.

38. The rebreathing oxygen supplementation device of claim 28, comprising:
a plurality of breakable regions within the enclosure, each of the breakable regions of a size and shape to position a section of tubing.

39. The rebreathing oxygen supplementation device of claim 28, comprising:
at least one attachment region for a gas input tube affixed to the enclosure.

40. The rebreathing oxygen supplementation device of claim 28, comprising:
an lower edge of the enclosure, the lower edge positioned to reversibly mate with the skin region of the patient at a position between a lower nose and a top lip of the patient.

41. The rebreathing oxygen supplementation device of claim 28, further comprising:
at least one tubular structure with a first end forming an aperture within the internal surface, and a second end forming an aperture distal to the enclosure.

42. The rebreathing oxygen supplementation device of claim 28, further comprising:
a tubular structure with a center region forming an aperture within the internal surface, and at least one second end forming an aperture distal to the enclosure.

43. The rebreathing oxygen supplementation device of claim 28, further comprising:
at least one attachment region for a gas input tube affixed to the enclosure; and
a gas emitter affixed to the at least one attachment region, the gas emitter positioned to direct gas into the nasal region of the patient.
